(19) United States
(12) Patent Application Publication
(10) Pub. No.: US 2007/0154546 A1
(43) Pub. Date: Jul. 5, 2007

(54) Sustained release pharmaceutical compositions

(76) Inventors: Jack Yongfeng Zhang, Diamond Bar, CA (US); Mary Ziping Luo, Diamond Bar, CA (US); Richard Li, Alhambra, CA (US); Jie Fei Ding, Fontana, CA (US)

Correspondence Address: CHRISTIE, PARKER & HALE, LLP, PO BOX 7068, PASADENA, CA, 91109-7068, US

(21) Appl. No.: 11/322,480

(22) Filed: Dec. 30, 2005

(51) Int. Cl. A61K 31/727; A61K 9/22; A61K 31/22; A61K 31/44

(52) U.S. Cl. 424/468; 514/56; 514/546; 514/358

(57) ABSTRACT

The present invention relates to a pharmaceutical composition comprising a salt of quaternary ammonium of an acid drug in the form of a suspension or an emulsion, suitable for parenteral administration and providing a sustained release of the drug. In one preferred embodiment, the present invention is directed to a long term sustained release composition for parenteral administration, comprising a salt of heparin or low molecular weight heparin in an emulsion with a salt of acylcholine, for the prevention and treatment of venous thrombosis and pulmonary embolism.

### BACKGROUND

Heparin and low molecular weight heparin (LMWH) are mixtures of polysaccharides of animal origin, containing acid groups and characterized by their anticoagulant and antithrombotic properties. They are mainly used for the prevention and treatment of venous thrombosis and pulmonary embolism during or after surgery. LMWH formulations are well known and widely used substances in clinical settings; examples include dalteparin under the name FRAGMIN®, enoxaparin under the name LOVENOX®, tinzaparin under the name INNOHEP® and several others.

In clinical use, heparin is administered through the parenteral route due to its poor oral bioavailability. In addition, it has a short half-life, resulting in the need for frequent dosing to maintain blood plasma concentration within the therapeutic range when the medication is needed for long term treatment. This proves to be inconvenient for patients, as they are often required to administer/receive several injections on a daily basis.

Furthermore, the currently available heparin products exhibit an undesirable drug release profile that shows a surge to a high peak blood plasma concentration above the therapeutic range and a rapid decline to a concentration below the therapeutic range. This results in a multiple “peaks and valleys” concentration profile when multiple injections are given several times a day over the course of days or weeks. LMWH, such as enoxaparin, has a longer half-life (4.5 hours) compared to heparin. This allows the administration frequency to be reduced to once or twice daily. However, the daily “peaks and valleys” concentration profile still exists. This will cause an increased occurrence of adverse effects such as bleeding complications and a high frequency of hemorrhagic accidents. Accordingly, a sustained release or long term sustained release composition of heparin or low molecular weight heparin that can extend the duration of effect over the course of several days would be very beneficial.

Sustained release technology has been widely used in pharmaceutical field. Sustained release drugs for oral administration through the digestion system usually have a duration of not more than 48 hours. On the other hand, sustained release drugs for injectable administration through intramuscular (IM) or subcutaneous (SC) entry may last much longer, sometimes up to months.

There are several known long-term sustained release technologies available for parenterally administered pharmaceuticals. These include the use of polymers, liposomes, gels and insoluble drugs or their salts or esters.

Polymer technology utilizes a method of encapsulating an active substance in a biodegradable polymer for sustained release of the pharmaceutical substance, or, alternatively, using the biodegradable polymer as a matrix to control the release of the substance. Examples of formulations using this type of technology include LUPRON DEPOT®, ZOLADEX®, and SANDOSTATIN LAR®

Liposome technology uses liposomes, a lipid based drug carrier, as the vehicle to carry and control the release of the active ingredients. Examples include DEPODUR® (previously known as Depo Morphine) and AMBISOME®

Gel technology utilizes a gel matrix to entrap the active substances and control the release rate of the substance. The gel based technology essentially utilizes a polymer that forms a thermally reversible hydrogel. The polymer can be injected as a liquid, gelling once it reaches body temperature, forming a gel matrix for sustained release of the active ingredient. The active substance can also be loaded into a lipid based drug carriers and sequestered into a gel matrix to further prolong the release of the active substance.

Finally, some pharmaceutical compositions use the low solubility characteristics of the active drugs, or its salts or esters. One example of this type of technology is medroxyprogesterone sold under the name DEPO-PROVERA®, with a solubility in human serum of 42 mcg/mL. A dose of 150 mg of Medroxyprogesterone given through IM administration can slowly dissolve and release for up to three months to form a long term sustained release.

Although sustained release technology has found a wide application in oral administration, it is often difficult to achieve sustained release for some medications in parenteral preparations, particularly for long-term sustained release. Many of the known methods for sustained release in parenteral preparations, moreover, are not entirely satisfactory in providing long-term sustained release, with safe active and inactive ingredients, the ability to maintain appropriate blood plasma or serum concentration levels of the drugs without “peaks and valleys”, and a high level of ease-of-use for both patients and healthcare professionals, particularly in providing safe and efficacious long-term sustained release pharmaceutical products for the prevention and treatment of venous. thrombosis.

### SUMMARY OF THE INVENTION

The present invention relates to a pharmaceutical composition for parenteral administration containing a salt of quaternary ammonium of an acid drug in the form of a suspension or an emulsion. In a preferred embodiment, the present invention is directed to a long term sustained release composition containing mixture of a salt of acid drugs and quaternary ammonium in the form of suspension or emulsion. The composition is suitable for parenteral administration through the intramuscular or subcutaneous route.

Thus, one embodiment of the present invention is directed to a sustained release pharmaceutical composition, comprising a quaternary ammonium salt of an acid drug.

Another embodiment of the present invention is directed to a sustained release pharmaceutical composition, comprising a quaternary ammonium salt of an acid drug, wherein the acid drug is an organic compound with one or more acid groups selected from the group consisting of carboxyl group, sulfonic group, sulfate group, and mixtures thereof. In a preferred embodiment, the acid drug is heparin or low molecular weight heparin. In some embodiments, the low molecular weight heparin is adreparin, certoparin, dalteparin, enoxaparin, nadroparin, parnaparin, reviparin, or tinzaparin.

Another embodiment of the present invention is directed to a sustained release pharmaceutical composition, comprising a quaternary ammonium salt of an acid drug, wherein the quaternary ammonium salt comprises at least eight carbon atoms. In one embodiment, the quaternary ammonium salt is selected from the group consisting of choline esters, alkylpyridines, and their derivatives. The use of choline esters as drug absorption enhancing agents is known from prior art, for example in U.S. Pat. No. 4,835,138. However, the use of choline esters in the present invention has an opposite function to provide sustained release of the acid drug as detailed further in this application.

Still another embodiment of the present invention is directed to a sustained release pharmaceutical composition, wherein the quaternary ammonium salt comprises a choline esterified with an aliphatic acid or its derivatives. In one embodiment, the aliphatic acid comprises from three to ten carbon atoms. In another embodiment, the aliphatic acid is selected from the group consisting of capric acid, nonanoic acid, octanoic acid, heptanoic acid, hexanoic acid, pentanoic acid, butyric acid, propionic acid, and mixtures thereof.

Still another embodiment of the present invention is directed to a sustained release pharmaceutical composition, wherein the quaternary ammonium salt comprises a choline esterified with a fatty acid or its derivatives. In one embodiment, the fatty acid comprises an even number of carbon atoms between twelve to twenty two. In still another embodiment, the fatty acid is selected from the group consisting of lauric acid, myristic acid, palmitic acid, stearic acid, arachidic acid, behenic acid, palmitoyleic acid, oleic acid, ricinoleic acid, linoleic acid, arachidonic acid, and mixtures thereof.

Another embodiment of the present invention is directed to a sustained release pharmaceutical composition, wherein the quaternary ammonium salt comprises a choline esterified with a phosphatidic acid or its derivatives.

Another embodiment of the present invention is directed to a sustained release pharmaceutical composition, wherein the quaternary ammonium salt of the acid drug has a solubility in human plasma or serum of less than or equal to about 50 mg/mL. In still another embodiment, the quaternary ammonium salt of an acid drug is suspended in an aqueous medium. In one embodiment, the quaternary ammonium salt has a particle size less than or equal to about 200 micrometer. In another embodiment, the aqueous medium comprises water for injection, U.S.P.

Another embodiment of the present invention is directed to a sustained release pharmaceutical composition comprising one or more inactive pharmaceutical excipients. In one embodiment, the excipient is selected from the group consisting of surfactants, suspending agents, dispersing agents, emulsifying agents, tonicity agents, preservatives, pH buffers, and mixtures thereof. In another embodiment, the composition has a pH from about 5.0 to about 9.5.

Still another embodiment of the present invention is directed to a sterile pharmaceutical aqueous suspension or emulsion composition for parenteral administration, comprising a salt obtained by reacting an acid drug with a quaternary ammonium compound, the resulting salt having a longer sustained release in vivo than the acid drug.

Another embodiment of the present invention is directed to a method for increasing the in vivo duration of a drug having an acid group, comprising the step of reacting the drug with a quaternary ammonium compound in an aqueous medium to form a salt.

### BRIEF DESCRIPTION OF THE DRAWINGS

FIG. 1 is a graph showing the in vivo profile of enoxaparin sodium, with anti-factor Xa as a marker, in rats (Example 1).

FIG. 2 is a graph showing the in vivo profile of enoxaparin caproylcholinium, with anti-factor Xa as a marker, in rats following subcutaneous injection (Example 2).

FIG. 3 is a graph showing the in vivo profile of enoxaparin lauroylcholinium, with anti-factor Xa as a marker, in rats following subcutaneous injection (Example 3).

FIG. 4 is a graph showing the in vivo profile of enoxaparin stearoylcholinium, with anti-factor Xa as a marker, in rats following subcutaneous injection (Example 4).

FIG. 5 is a graph showing the in vivo profile of enoxaparin caproylcholinium, with anti-factor Xa as a marker, in rats following intramuscular injection (Example 5).

FIG. 6 is a graph showing the in vivo profile of enoxaparin lauroylcholinium, with anti-factor Xa as a marker, in rats following intramuscular injection (Example 6).

FIG. 7 is a graph showing the in vivo profile of enoxaparin myristoylcholinium, with anti-factor Xa as a marker, in rats following intramuscular injection (Example 7).

FIG. 8 is a graph showing the in vivo profile of enoxaparin palmitoylcholinium, with anti-factor Xa as a marker, in rats following intramuscular injection (Example 8).

FIG. 9 is a graph showing the in vivo profile of enoxaparin stearoylcholinium, with anti-factor Xa as a marker, in rats following intramuscular injection (Example 9).

FIG. 10 is a graph showing the profile of enoxaparin acylcholinium solubility in human serum as a function of the number of carbon atoms in the fatty acids.

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the compositions and methodologies that are described in the publications which might be used in connection with the presently described invention. The publications discussed above, below and throughout the text are provided solely for their disclosure prior to the filing date of the present application. Nothing herein is to be construed as an admission that the inventors are not entitled to antedate such disclosure by virtue of prior invention.

The present invention relates generally to a long-term sustained release pharmaceutical composition for parenteral administration comprising a salt of quaternary ammonium of an acid drug in the form of a suspension or an emulsion. More particularly, the pharmaceutical composition of the present invention is a sustained release composition comprising a mixture of salts of radicals of acid drugs and salts of quaternary ammonium suspended in an aqueous solution. In a preferred embodiment, the present invention is directed to a long term sustained release composition containing mixture of a salt of acid drugs and quaternary ammonium in the form of suspension or emulsion for the prevention and treatment of venous thrombosis. The composition is suitable for parenteral administration through the intramuscular or subcutaneous route.

Acid drugs suitable for use with the practice of this invention include, but are not limited to, organic compounds containing one or more acid groups such as carboxyl groups, sulfonic groups, sulfate groups, or any other acid radicals and their combinations thereof. For preparing compositions useful for the prevention and treatment of venous thrombosis and pulmonary embolism, an example of the acid drugs utilized in the composition of the present invention include heparin or low molecular weight heparin (LMWH) containing carboxyl and sulfate groups. Another example of an acid drug suitable for practice of the present invention is Montelukast, with a carboxyl group.

Examples of quaternary ammonium suitable for practice of the present invention include, but are not limited to, choline esters and alkylpyridines, and their derivatives. Preferred choline esters are acylcholine and esters of cholines with aliphatic acids, fatty acids, phosphatidic acids or their derivatives. Suitable aliphatic acids include, but not limited to, capric acid, nonanoic acid, octanoic acid, heptanoic acid, hexanoic acid, pentanoicacid, butyric acid, and propionic acid. Suitable fatty acids include, but not limited to, lauric acid, myristic acid, palmitic acid, stearic acid, arachidic acid, behenic acid, palmitoylleic acid, oleic acid, ricinoleic acid, linoleic acid, arachidonic acid.

Optionally, the composition of the present invention may also contain various inactive pharmaceutical excipients. The excipients may include one or more of surfactants, suspending agents, dispersing agents, emulsifying agents, tonicity agents, preservatives, pH buffers, and the like, as will be appreciated by one of skill in the art. Examples of excipients that can be utilized with the composition of the present invention include, but are not limited to, surfactants such as polysorbate, polozamer, polyethylene sorbitan fatty acid esters, lecithin as well as any natural or synthesized surfactants. The composition may also include a buffer, preferably to adjust the pH of the composition to a pH from about 5.0 to about 9.5.

A preferred composition of the present invention is a long-term sustained release mixture comprising a) salt(s) of acid drugs, b) salt(s) of acylcholines, and c) one or more inactive pharmaceutical excipients, suspended in an aqueous solution in the form of either a suspension or an emulsion. The salts preferably have particle size not greater than about 200 micrometer. The aqueous solution utilized in the present invention is preferably water for injection U.S.P.

As will be appreciated by the skilled artisan, the compositions of present invention may be prepared and supplied in several different ways. (1) In one embodiment, the salts are pre-compounded and provided as an emulsion or suspension in pre-filled syringes, vials, or similar containers. (2) In another embodiment, a solution of soluble salts of acid drugs is provided separately with a solution of soluble salts of acylcholines or other quaternary ammonium salt. Users then combine the two solutions prior to use. (3) In still another embodiment, either or both of the salts is supplied in a dry state (powder for injection) and solubilized in water or other aqueous medium by the user prior to use.

The sustained release composition of the present invention has a prolonged duration of effect that lasts for approximately 24 to 96 hours or more depending on the dose administered, the mode of administration, and the number of carbon atoms or the type of cholinium selected. The sustained release composition of the present invention contains a microparticle suspension, allowing the active ingredient to be slowly released at a gradual rate, thereby providing extended duration of effect by inhibiting systemic drug absorption and maintaining the drug concentration within the therapeutic range for a longer period of time. This proves to be beneficial as it avoids daily high peak concentration that can cause side effects and it also allows less frequent administration.

Various embodiment of the sustained release composition of the present invention can possess several advantages over prior art compositions, including:

1. Maintaining a more normal blood plasma or serum concentration level of the drugs. For example, blood potency of antifactor Xa from heparin or low molecular weight heparin, which is normal for both efficacy and safety consideration, is within the range of 0.1 to 1.1 IU/mL. (Goodman and Gilman's Pharmacological Basis of Therapeutics, p. 1344.) It has been shown that for a 5 day regimen of 1.5 mg/kg one per day SC dose of enoxaparin sodium, the mean Aₘₐₓ for antifactor Xa is 1.4 IU/mL, higher than the normal blood potency. (Lovenox, Enoxaparin Sodium Injection, package insert, July 2004.) One or more embodiments of the inventive composition, by contrast, provide for sustained release and avoid the daily peaks and valleys associated with daily dose of immediate release formulations, which can be potentially harmful or below the efficacy threshold for patients.

2. Providing convenience for both patients and healthcare professionals. Daily injections (or more; for example, some regimens for treating, for example, venous thrombosis and pulmonary embolism, call for dosing every 12 hours with 30 mg enoxaparin, or even every 8 hours with 5000 U heparin) cause inconvenience for both patients and nurses. One or more embodiments of the inventive composition, with its sustained release of the therapeutic agent, can be injected once every three to five days for acylcholinium enoxaparin or even longer time for other drugs. For example, prior to the present invention, patients are required to receive daily in-hospital injections of enoxaparin one week prior to undergoing brain surgery, but using a composition according to one or more embodiments of the present invention, one or two injections before the surgery is sufficient.

3. Acylcholinium compounds are inactive ingredients in a composition of the present invention. These compounds help to reach a suitable drug solubility and provide the desired long-term sustained release for the drug. The safety for an inactive ingredient is an important consideration in drug formulation development. In human bodies, acylcholinium, for fatty acids hydrolyzed as fatty acids, and choline are both safe compounds and naturally exist in human bodies in large amounts. (McMurry J., Organic Chemistry 5^{th} edition, p. 1120; United States Department of Agriculture, Agricultural Research service, Folate and Choline Interplay Investigated.)

### EXAMPLES

Examples provided are intended to assist in a further understanding of the invention. Particular materials employed, species and conditions are intended to be further illustrative of the invention and not limiting of the reasonable scope thereof.

Example 1 illustrates the in vivo profile (in a rat model) of currently marketed immediate release LMWH drug, LOVENOX®, and is presented for comparison purposes.

Examples 2 through 9 illustrate: (1) the preparation of various esters of choline, (2) preparation of enoxaparin acylcholinium, (3) in vitro solubility of enoxaparin acylcholinium and (4) in vivo sustained release profile of enoxaparin acylcholinium, in a rat model.

Example 10 shows the preparation of enoxaparin cetyl-pyridinium and its in vitro solubility in human serum.

Examples 11 and 12 demonstrate the preparation of montelukast acylcholinium and the in vitro solubility thereof.

Example 13 demonstrates the preparation of montelukast cetyl-pyridinium and its in vitro solubility.

The therapeutically effective range for the LMWH (enoxaparin) is considered to be between 0.1 to 1.1 IU/mL of anti-factor Xa in serum. (Antifactor Xa is a marker for heparin or LMWH in blood plasma). In these examples, 0.2 IU/mL of anti-factor Xa was used as the criterion for evaluation of sustained release duration.

In the following examples, the acylcholine salt with the following structure:
[RCOOC₂H₄N^{⊕}(CH₃)₃]ₙE^{n⊖}
was generally named as acylcholinium for acid drug E, where:
E is an acid drug with carboxyl groups, sulfonic groups, sulfates and other acid radicals or their combinations, such as low molecular weight heparin, e.g. enoxaparin; and
n is the valence number of the acid drug E.

Specifically, where E=enoxaparin:
R=C₉H₁₉, is enoxaparin caproylcholinium
R=C₁₁H₂₃, is enoxaparin lauroylcholinium
R=C₁₃H₂₇, is enoxaparin myristoylcholinium
R=C₁₅H₃₁, is enoxaparin palmitoylcholinium
R=C₁₇H₃₅, is enoxaparin stearoylcholinium

### Example 1 (Prior Art)

Enoxaparin sodium is the major LMWH in the current market. Its saturation solubility in water is about 30% or 300 mg/mL. This example illustrates the in vivo profile of enoxaparin sodium, with anti-factor Xa as marker, in rats. Thirteen (13) Sprague-Dawley rats, body weight about 250 g, were injected with approximately 1 mg or 100 IU of LOVENOX® (per anti-factor Xa) through subcutaneous administration. After injection, the blood sample was withdrawn within an 8 hours period. Anti-factor Xa in rat serum samples was analyzed by an amidolytic assay method adapted from European Pharmacopeia (EP). The tested results are summarized in Table 1, below.

TABLE 1
Xa in Rat Serum for Example 1, Enoxaparin Sodium, 1 mg, SC

| | | | | | | | | | |
|---|---|---|---|---|---|---|---|---|---|
| Time | 0 | 0.5 | 1 | 1.5 | 2 | 3 | 4 | 6 | 8 |
| (Hour) | | | | | | | | | |
| Xa | 0.00 | 1.86 | 2.30 | 2.32 | 2.18 | 1.70 | 1.18 | 0.53 | 0.19 |
| (IU/mL) | | | | | | | | | |

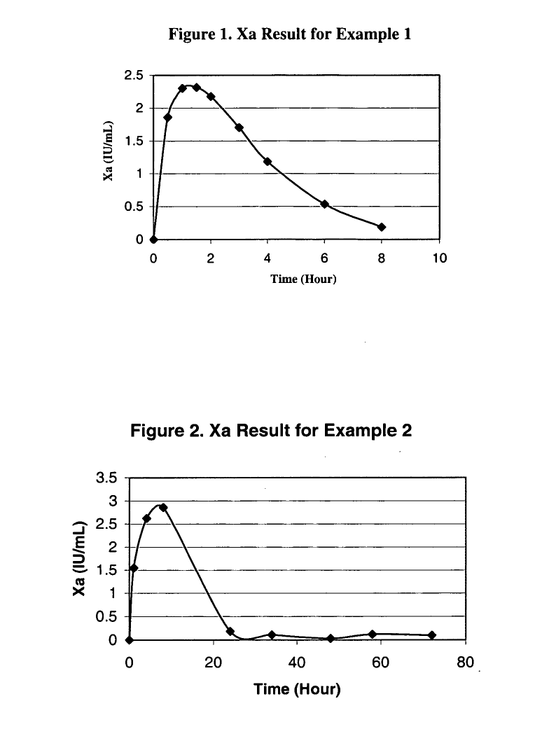

This study shows the profile of the currently available immediate release enoxaparin sodium, LOVENOX®. The Aₘₐₓ of 2.3 IU/mL appeared at 1.5 hour, the half life (t_{1/2}) was about 3 hours. The effective duration of Xa, defined as Xa in rat serum>0.2 IU/mL, was about 8 hours. The curve of mean anti-factor Xa concentration in rat serum versus time is shown in FIG. 1.

### Example 2

This example illustrates the preparation of enoxaparin caproylcholinium and its in vivo profile in a rat model.

(1) Preparation of caproylcholinium chloride:
Caproylcholinium chloride is choline caprate chloride with the following structure:
   [C₉H₁₉COOC₂H₄N^{⊕}(CH₃)₃]Cl
Synthesis process:
   C₉H₁₉COOH+ClC₂H₄OH→C₉H₁₉COOC₂H₄Cl+H₂O
   C₉H₁₉COOC₂H₄Cl+N(CH₃)₃→[C₉H₁₉COOC₂H₄N⁺(CH₃)₃]Cl⁻
Seven grams (7.0 g) of 2-chloroethyl alcohol was added to a solution of capric acid (10.0 g) and sulfuric acid (0.34 mL). The obtained mixture was heated to a temperature of 95-105° C. and maintained for 1.5 hours. After extraction by ethylacetate and concentration, the crude capric acid 2-chloroethyl ester was obtained. The crude ester (10.0 g) and trimethylamine (7.5 g) were dissolved in DMF (17.5 mL). The mixture was incubated at 75-80° C. for 8-9 hours followed by a separation and drying process to obtain 9 g of caproylcholinium chloride.

Another synthesis process:
C₉H₁₉COCl+(CH₃)₃N⁺C₂H₄OH Cl⁻→[C₉H₁₉COOC₂H₄N⁺(CH₃)₃]Cl⁻+HCl
Ten grams (10.0 g) of choline chloride were added caproyl chloride (13.7 g). The mixture was heated to a temperature of 110° C. and maintained for 30 minutes. After cooling, the mixture was washed by ethylacetate. The solid was then collected using a filter and dried to obtain 20 g of Caproylcholinium chloride.

(2) Preparation of enoxaparin caproylcholinium:
One (1) mL of aqueous solution containing 40 mg/ml of enoxaparin sodium and 10 mg/ml of polysorbate 80 was prepared and mixed with 1 mL of another aqueous solution containing 64 mg/ml of choline caprate chloride. After mixing and shaking, a suspension formed. The process to generate enoxaparin caproylcholinium is:
   nQ⁺Cl⁻+ENaₙ→Q⁺ₙEⁿ⁻+n NaCl
where Q⁺=C₉H₁₉COOC₂H₄N⁺(CH₃)₃, enoxaparin sodium is simplified as ENaₙ and n is the valence number of the enoxaparin molecules. The solid salt of enoxaparin caproylcholinium can be obtained through centrifugation.

(3) In vitro Solubility.
Enoxaparin caproylcholinium shows a solubility of 25.2±2.6 mg/mL as enoxaparin sodium in human serum.

(4) In vivo Sustained Release Profile for Rat Model.
Six (6) Sprague-Dawley rats, with body weight about 250 g, were injected with approximately 0.3 ml of the mixed solution, which contained 6 mg, or 600 IU, of enoxaparin sodium (per anti-factor Xa), through subcutaneous administration.

The rat serum samples were analyzed using the same method as described in Example 1. The tested results are summarized below in Table 2. Enoxaparin caproylcholinium shows AUC of 47 IU/mL hr and a longer duration of anti-factor Xa, about 24 hours. See FIG. 2.

TABLE 2
Xa in Rat Serum for Example 2, 6 mg, SC

| | | | | | | | | | |
|---|---|---|---|---|---|---|---|---|---|
| Time | 0 | 1 | 4 | 8 | 24 | 34 | 48 | 58 | 72 |
| (Hour) | | | | | | | | | |
| Xa | 0.00 | 1.55 | 2.62 | 2.86 | 0.19 | 0.11 | 0.04 | 0.12 | 0.10 |
| (IU/mL) | | | | | | | | | |

### Example 3

This example illustrates the preparation of enoxaparin lauroylcholinium and its in vivo profile in a rat model.

(1) Preparation of Lauroylcholinium chloride:
Lauroylcholinium chloride is choline laurate chloride with the following structure:
   [C₁₁H₂₁COOC₂H₄N⁺(CH₃)₃]Cl⁻
The synthesis process:
   C₁₁H₂₃COOH+ClC₂H₄OH→C₁₁H₂₃COOC₂H₄Cl+H₂O
   C₁₁H₂₃COOC₂R₄Cl+N(CH₃)₃→[C₁₁H₂₃COOC₂H₄N⁺(CH₃)₃]Cl⁻
Six grams (6.0 g) of 2-chloroethyl alcohol was added to a solution of lauric acid (10.0 g) and sulfuric acid (0.32 mL). The mixture was heated to a temperature of 95-105° C. and maintained for 1.5 hours. After extraction by ethylacetate and concentration, the crude lauric acid 2-chloroethyl ester was obtained. The crude ester (10.0 g) and trimethylamine (6.7 g) were dissolved in DMF (16.7 mL). The mixture was incubated at 75-80° C. for 8-9 hours followed by a separation and drying process to obtain 9 g of lauroylcholinium chloride.

(2) Preparation of enoxaparin lauroylcholinium:
One (1) mL of aqueous solution containing 40 mg/ml of enoxaparin sodium and 10 mg/ml of polysorbate 80 was prepared and mixed with 1 mL of another aqueous solution containing 72 mg/ml of choline laurate chloride. After mixing and shaking, a suspension solution was formed.
The process to generate enoxaparin lauroylcholinium is
   nQ⁺Cl⁻+ENaₙ→Q⁺ₙEⁿ⁻+nNaCl
where Q⁺=C₁₁H₂₃COOC₂H₄N⁺(CH₃)₃, enoxaparin sodium is simplified as ENaₙ and n is the valence number of the enoxaparin molecules. The solid salt of enoxaparin lauroylcholinium can be obtained through centrifugation.

(3) In vitro Solubility.
Enoxaparin lauroylcholinium shows a solubility of 13.6±2.2 mg/mL as enoxaparin sodium in human serum.

(4) In vivo Sustained Release Profile for Rat Model.

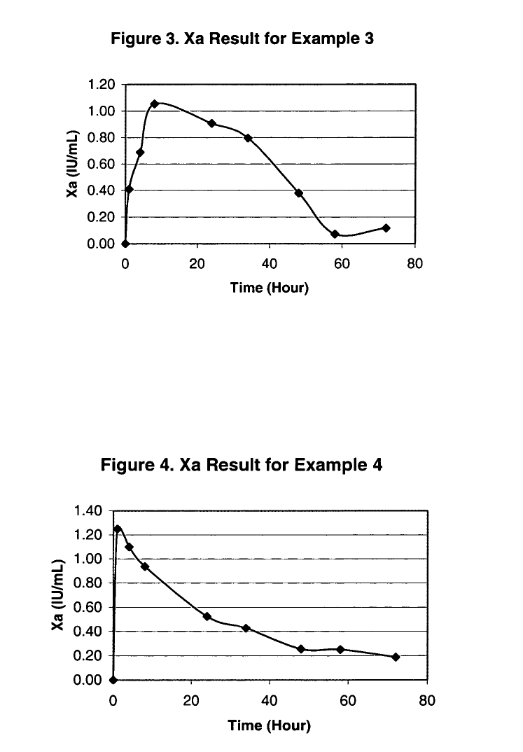

Six (6) Sprague-Dawley rats, with body weight about 250 g, were injected with approximately 0.3 ml of the suspension obtained above, which contained 6 mg, or 600 IU, of enoxaparin sodium (per anti-factor Xa), through subcutaneous administration. The rat serums samples were analyzed using the same method as Example 1. The tested results are summarized below in Table 3. Enoxaparin lauroylcholinium shows an AUC of 41 IU/mL hr and a longer duration of anti-factor Xa, about 54 hours. See FIG. 3.

TABLE 3
Xa in Rat Serum for Example 3, 6 mg, SC

| | | | | | | | | | |
|---|---|---|---|---|---|---|---|---|---|
| Time | 0 | 1 | 4 | 8 | 24 | 34 | 48 | 58 | 72 |
| (Hour) | | | | | | | | | |
| Xa | 0.00 | 0.41 | 0.69 | 1.05 | 0.91 | 0.80 | 0.38 | 0.07 | 0.12 |
| (IU/mL) | | | | | | | | | |

### Example 4

This example illustrates the preparation of enoxaparin stearoylcholinium and its in vivo profile in a rat model.

(1) Preparation of stearoylcholinium chloride:
Stearoylcholinium chloride is choline stearate chloride with the following structure:
   [C₁₇H₃₅COOC₂H₄N⁺(CH₃)₃]Cl⁻
The synthesis process:
   C₁₇H₃₅COOH+ClC₂H₄OH→C₁₇H₃₅COOC₂H₄Cl+H₂O
   C₁₇H₃₅COOC₂H₄Cl+N(CH₃)₃→[C₁₇H₃₅COOC₂H₄N⁺(CH₃)₃]Cl⁻
4.2 gram of 2-chloroethyl alcohol was added to a solution of stearic acid (10.0 g) and sulfuric acid (0.28 mL). The mixture was heated to a temperature of 95-105° C. and maintained for 1.5 hours. After extraction by ethylacetate and concentration, the crude stearic acid 2-chloroethyl ester was obtained. The crude ester (10.0 g) and trimethylamine (5.0 g) were dissolved in DMF (13.0 mL). The mixture was incubated at 75-80° C. for 8-9 hours followed by a separation and drying process to obtain 9 g of stearoylcholinium chloride.

(2) Preparation of enoxaparin stearoylcholinium
A mixture was prepared by adding 50 ml of 20 mg/ml enoxaparin sodium solution to 50 ml of a solution containing 52 mg/ml choline stearate chloride and 20 mg/ml polysorbate 80. The mixture was held for a while and some supernatant was collected from the mixed solution to make a solution containing about 22 mg/ml of enoxaparin sodium and to form the suspension. The process to generate enoxaparin stearoylcholinium is
   nQ⁺Cl⁻+ENaₙ→Q⁺ₙEⁿ⁻+nNaCl
where Q⁺=C₁₇H₃₅COOC₂H₄N⁺(CH₃)₃, enoxaparin sodium is simplified as ENaₙ and n is the valence number of the enoxaparin molecules. The solid salt of enoxaparin stearoylcholinium can be obtained through centrifugation.

(3) In vitro Solubility.
Enoxaparin stearoylcholinium shows a solubility of 1.5±0.3 mg/mL as enoxaparin sodium in human serum.

(4) In vivo Profile for Rat Model.

Four (4) Sprague-Dawley rats, with body weight about 250 g, were injected with approximately 0.3 ml of suspension solution obtained above, which contained 6.6 mg, or 660 IU, of enoxaparin sodium (per anti-factor Xa), through subcutaneous administration. The rat serums samples were analyzed using the same method as Example 1. The results are summarized below in Table 4. Enoxaparin stearoylcholinium shows an AUC of 35 IU/mL hr and a longer duration of anti-factor Xa, about 57 hours. See FIG. 4.

TABLE 4
Xa in Rat Serum for Example 4, 6.6 mg, SC

| | | | | | | | | | |
|---|---|---|---|---|---|---|---|---|---|
| Time | 0 | 1 | 4 | 8 | 24 | 34 | 48 | 58 | 72 |
| (Hour) | | | | | | | | | |
| Xa | 0.00 | 1.25 | 1.10 | 0.94 | 0.53 | 0.43 | 0.26 | 0.25 | 0.19 |
| (IU/mL) | | | | | | | | | |

### Example 5

This example illustrates the preparation of enoxaparin caproylcholinium and its in vivo profile in a rat model. Preparation of the caproylcholinium chloride is the same as in example 2.

(1) Preparation of the enoxaparin caproylcholinium.

Twenty five (25) ml of 20 mg/ml enoxaparin sodium solution was added to 25 ml of solution containing 38 mg/ml choline caprate chloride and 20 mg/ml polysorbate 80. The mixture was stabilized for a while to allow it to settle. A portion of the supernatant was collected from the mixed solution to make a solution containing 22 mg/ml enoxaparin sodium. The suspension was formed and ready to use for in vivo study.

(2) In vivo Profile for Rat Model.

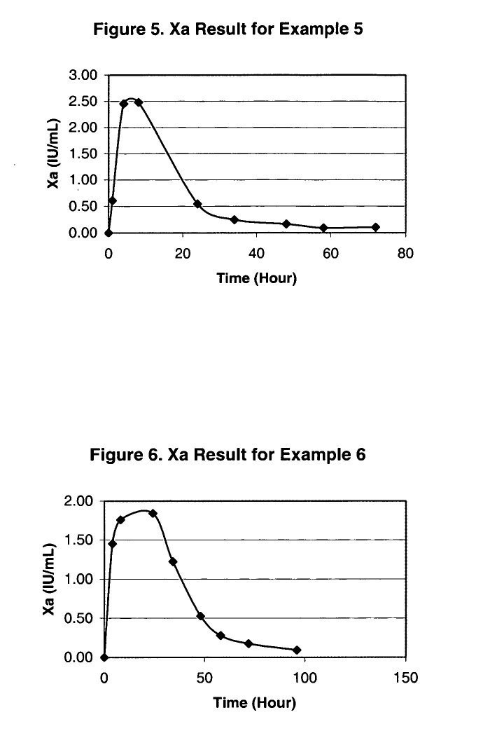

Six (6) Sprague-Dawley rats, with body weight about 250 g, were injected with approximately 0.3 ml of the mixed solution, which contained 6.6 mg, or 660 IU, of enoxaparin sodium (per anti-factor Xa) through IM administration. The rat serum samples were analyzed using the same method as Example 1. The results are summarized below in Table 5. Enoxaparin caproylcholinium shows an AUC of 49 IU/mL hr and a longer duration of anti-factor Xa, about 42 hours. See FIG. 5.

TABLE 5
Xa in Rat Serum for Example 5, 6.6 mg, IM

| | | | | | | | | | |
|---|---|---|---|---|---|---|---|---|---|
| Time | 0 | 1 | 4 | 8 | 24 | 34 | 48 | 58 | 72 |
| (Hour) | | | | | | | | | |
| Xa | 0.00 | 0.61 | 2.45 | 2.48 | 0.55 | 0.25 | 0.16 | 0.09 | 0.10 |
| (IU/mL) | | | | | | | | | |

### Example 6

This example illustrates the preparation of enoxaparin lauroylcholinium and its in vivo profile in a rat model. Preparation of the lauroylcholinium chloride is the same as in example 3.

(1) Preparation of enoxaparin lauroylcholinium.
One (1) g enoxaparin sodium, 2.07 g choline laurate chloride, 1 g polysorbate 80 and 1 g sodium chloride were dissolved in 20 ml of water. The solution was heated to 50° C., 80 ml of water was added and the solution was mixed and held for a while to allow settlement. A portion of the supernatant was collected from the solution to form a suspension solution containing 22 mg/ml of enoxaparin sodium, which was ready for use in the in vivo study.

In vivo Profile for Rat Model.

Five (5) Sprague-Dawley rats, with body weight about 250 g, were injected with approximately 0.3 ml of the mixed solution, which contained 6.6 mg, or 660 IU, of enoxaparin sodium (per anti-factor Xa), through IM administration. The rat serum samples were analyzed using the same method as Example 1. The tested results are summarized below in Table 6. Enoxaparin lauroylcholinium shows an AUC of 76 IU/mL hr and a longer duration of Anti-factor Xa, about 69 hours. See FIG. 6.

TABLE 6
Xa in Rat Serum for Example 6, 6.6 mg, IM

| | | | | | | | | | |
|---|---|---|---|---|---|---|---|---|---|
| Time | 0 | 4 | 8 | 24 | 34 | 48 | 58 | 72 | 96 |
| (Hour) | | | | | | | | | |
| Xa | 0.00 | 1.45 | 1.76 | 1.84 | 1.22 | 0.53 | 0.28 | 0.17 | 0.09 |
| (IU/mL) | | | | | | | | | |

### Example 7

This example illustrates the preparation of enoxaparin myristoylcholinium and its in vivo profile in a rat model.

(1) Preparation of enoxaparin myristoylcholinium. 0.9 g enoxaparin sodium, 2.0 g choline myristate chloride, 3 g polysorbate 80 and 2 g sodium chloride were dissolved in 40 ml of water. The solution was heated to 50° C., 160 ml of water was added and the solution was mixed and held for a while to allow it to settle. A portion of the supernatant was collected from the solution to form a suspension solution containing 22 mg/ml of enoxaparin sodium.

The process to generate enoxaparin myristoylcholinium is
nQ⁺Cl⁻+ENaₙ→Q⁺ₙEⁿ⁻+nNaCl
where Q⁺=C₁₃H₂₇COOC₂H₄N⁺(CH₃)₃, enoxaparin sodium is simplified as ENaₙ and n is the valence number of the enoxaparin molecules. The solid salt of enoxaparin myristoylcholinium can be obtained through centrifugation.

(2) In vitro Solubility.
Enoxaparin myristoylcholinium shows a solubility of 5.2±2.0 mg/mL as enoxaparin sodium in human serum.

(3) In vivo Profile for Rat Model.

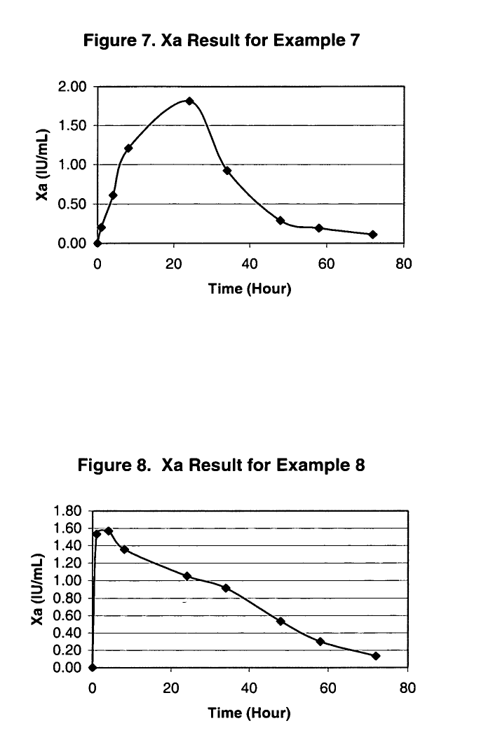

Six (6) Sprague-Dawley rats, with body weights about 250 g, were injected with approximately 0.3 ml of the mixed solution, which contained 6.6 mg, or 660 IU, of enoxaparin sodium (per anti-factor Xa), through IM administration. The rat serum samples were analyzed using the same method as Example 1. The results are summarized below in Table 7. Enoxaparin myristoylcholinium has an AUC of 56 IU/mL hr and a longer duration of Anti-factor Xa, about 57 hours. See FIG. 7.

TABLE 7
Xa in Rat Serum for Example 7, 6.6 mg, IM

| | | | | | | | | | |
|---|---|---|---|---|---|---|---|---|---|
| Time | 0 | 1 | 4 | 8 | 24 | 34 | 48 | 58 | 72 |
| (Hour) | | | | | | | | | |
| Xa | 0.00 | 0.20 | 0.61 | 1.21 | 1.82 | 0.92 | 0.29 | 0.19 | 0.11 |
| (IU/mL) | | | | | | | | | |

### Example 8

This example illustrates the preparation of enoxaparin palmitoylcholinium and its in vivo profile in a rat model.

(1) Preparation of Palmitoylcholinium chloride.
Palmitoylcholinium chloride is choline palmitate chloride with the following structure:
   [C₁₅H₃₁COOC₂H₄N⁺(CH₃)₃]Cl⁻
The synthesis process:
   C₁₅H₃₁COOH+ClC₂H₄OH→C₁₅H₃₁COOC₂H₄Cl+H₂O
   C₁₅H₃₁COOC₂R₄Cl+N(CH₃)₃→[C₁₅H₃₁COOC₂H₄N⁺(CH₃)₃]Cl⁻
4.7 grams 2-chloroethyl alcohol was added to a solution containing palmitic acid (10.0 g) and sulfuric acid (0.29 mL). The mixture was heated to a temperature of 95-105° C. and maintained for 1.5 hours. After extraction by ethylacetate and concentration, the crude palmitic acid 2-chloroethyl ester obtained. The crude ester (10.0 g) and trimethylamine (5.5 g) were dissolved in DMF (15.5 mL). The mixture was incubated at 75-80° C. for 8-9 hours followed by separation and drying process to obtain 9 g palmitoylcholinium chloride.
Another synthesis process:
   C₁₅H₃₁COCl+(CH₃)₃N+C₂H₄OH Cl⁻→[Cl₁₅H₃₁COOC₂H₄N⁺(CH₃)₃]Cl⁻+HCl
Thirty grams (30.0 g) choline chloride was added to a solution of palmitoyl chloride (63.0 g). The mixture was heated to a temperature of 120° C. and maintained for 30 minutes. After cooling to room temperature, the mixture was washed with ethylacetate. The solids were collected using a filter and dried to obtain 86 g of palmitoylcholinium chloride.

(2) Preparation of Enoxaparin Palmitoylcholinium.
Solution A was prepared by dissolving 2.0 g of enoxaparin sodium in 25 mL of water. Solution B was prepared by dissolving 3.5 g of choline palmitate chloride in 25 mL of water. Solution A and solution B were then added to a 150 ml solution containing 2.0 g polysorbate 80. The solution was mixed and held for a short period of time and then a portion of supernatant was collected. The suspension solution containing 16 mg/ml of enoxaparin sodium was formed. The process to generate enoxaparin palmitoylcholinium is:
   nQ⁺Cl⁻+ENaₙ→Q⁺ₙEⁿ⁻+nNaCl
where Q⁺=C₁₅H₃₁COOC₂H₄N⁺(CH₃)₃, enoxaparin sodium is simplified as ENaₙ and n is the valence number of the enoxaparin molecules. The solid salt of enoxaparin palmitoylcholinium can be obtained through centrifugation.

(2) In vitro Solubility.
Enoxaparin palmitoylcholinium has a solubility of 3.6±1.2 mg/mL as enoxaparin sodium in human serum.

(3) In vivo Profile for Rat Model.

Six (6) Sprague-Dawley rats, with body weights about 250 g, were injected with approximately 0.3 ml of the suspension obtained above, which contained 4.8 mg, or 480 IU, of enoxaparin sodium (per anti-factor Xa) through IM administration. The rat serums samples were analyzed using the same method as Example 1. The results are summarized below in Table 8. Enoxaparin palmitoylcholinium shows an AUC of 58 IU/mL hr and a longer duration of Anti-factor Xa, about 66 hours. See FIG. 8.

TABLE 8
Xa in Rat Serum for Example 8, 4.8 mg, IM

| | | | | | | | | | |
|---|---|---|---|---|---|---|---|---|---|
| Time | 0 | 1 | 4 | 8 | 24 | 34 | 48 | 58 | 72 |
| (Hour) | | | | | | | | | |
| Xa | 0.00 | 1.53 | 1.57 | 1.36 | 1.05 | 0.92 | 0.53 | 0.30 | 0.13 |
| (IU/mL) | | | | | | | | | |

### Example 9

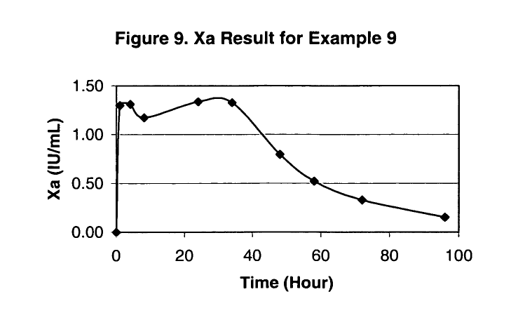

This example illustrates the in vivo profile of enoxaparin stearoylcholinium in a rat model. This example is the same as Example 4, except the administration is through IM instead of SC. The rat serum samples were analyzed using the same method as in Example 1. The results are summarized below in Table 9. Enoxaparin stearoylcholinium shows an AUC of 76 IU/mL hr and a longer duration of Anti-factor Xa, about 90 hours. See FIG. 9.

TABLE 9
Xa in Rat Serum for Example 9, 6.6 mg, IM

| | | | | | | | | | | |
|---|---|---|---|---|---|---|---|---|---|---|
| Time (Hour) | 0 | 1 | 4 | 8 | 24 | 34 | 48 | 58 | 72 | 96 |
| Xa (IU/mL) | 0.00 | 1.30 | 1.31 | 1.17 | 1.34 | 1.33 | 0.80 | 0.52 | 0.33 | 0.15 |

### Example 10

This example illustrates the preparation of enoxaparin cetyl-pyridinium and its solubility in human serum.

Preparation of Enoxaparin Cetyl-pyridinium. Solution A was prepared by dissolving 0.5 g enoxaparin sodium and 0.25 g polysorbate 80 in 25 mL of water. Solution B was prepared by dissolving 1.0 g cetyl-pyridinium chloride in 25 mL of water. The two solutions were then mixed and held for a short period of time. The solid salt of enoxaparin cetyl-pyridinium was obtained through centrifugation. The process to generate enoxaparin cetyl-pyridinium is:
n[C₁₆H₃₃—C₅H₅N⁺]Cl⁻+ENaₙ→[C₁₆H₃₃—C₅H₅N⁺]ₙEⁿ⁻+n NaCl
where enoxaparin sodium is simplified as ENaₙ, and where n is the valence number of the enoxaparin molecules. Enoxaparin cetyl-pyridinium shows a solubility of 0.08 mg/mL as enoxaparin sodium in human serum.

### Example 11

Montelukast is also an acid drug. It is described chemically as [R-(E)-1-[[[1-[3-[2-(7-choloro-2-quinolinyl)ethenyl]phenyl]-3-[2(1-hyroxy-methylethyl) phenyl] propyl] thio]methyl] cyclopropaneacetic acid, monosodium salt. It has the following chemical structure:

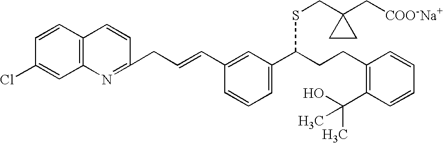

Montelukast contains a carboxyl group. Montelukast sodium, C₃₄H₃₅ClNSCOONa, is normally used to cure asthma, for immediate release. Montelukast sodium is very easy to dissolve in water, with a solubility in water of more than 100 mg/mL. This example illustrates the preparation of montelukast lauro-cholinium and its solubility in human serum.

Preparation of Montelukast Lauro-cholinium. Solution A (10% montelukast sodium in ethanol) and solution B (10% choline laurate chloride in DI water) were prepared. A mixture of 0.3 mL solution A and 0.158 mL solution B was prepared, 1.1 mL of water was added, and the mixture allowed to sit for a while. The salt of montelukast lauro-cholinium was obtained through centrifugation. The process to generate montelukast lauro-cholinium is:
[C₁₁H₂₃COOC₂H₄N⁺(CH₃)₃]Cl⁻+C₃₄H₃₅ClNSCOONa→[C₁₁H₂₃COOC₂H₄N⁺(CH₃)₃]C₃₄H₃₅Cl NSCOO⁻+NaCl
Montelukast laurocholinium has a solubility of 2.8 mg/mL in human serum, calculated as montelukast sodium. It was measured by an HPLC method.

### Example 12

This example illustrates the preparation of montelukast palmito-cholinium and measurement of its solubility in human serum.

Preparation of montelukast palmito-cholinium. Solution A (containing 10% montelukast sodium in ethanol) and solution B (containing 5% choline palmitic chloride in DI water) were prepared. Solution A (0.3 mL) was mixed with 0.372 mL solution B, then 0.9 mL water was added and the solution allowed to sit for a while. The salt of montelukast palmito-cholinium was obtained through centrifugation. The process to generate Montelukast palmito-cholinium is:
[C₁₅H₃₁COOC₂H₄N⁺(CH₃)₃]Cl⁻+C₃₄H₃₅ClNSCOONa→[C₁₅H₃₁COOC₂H₄N⁺(CH₃)₃]C₃₄H₃₅Cl NSCOO⁻+NaCl

Montelukast palmito-cholinium shows a solubility of 1.6 mg/mL in human serum, calculated as montelukast sodium. It was measured by an HPLC method.

### Example 13

This example illustrates the preparation of montelukast cetyl-pyridinium and its solubility in human serum.

Preparation of Montelukast Cetyl-pyridinium. Solution A (containing 5% montelukast sodium in DI water) and solution B (containing 10% of Cetyl-pyridinium chloride in DI water) were prepared. 0.6 mL of solution A was mixed with 0.177 mL of solution B and allowed to sit. The salt of montelukast cetyl-pyridinium was obtained through centrifugation. The process to generate Montelukast Cetyl-pyridinium is:
[C₂₁H₃₈N⁺]Cl⁻+C₃₄H₃₅ClNSCOONa→[C₂₁H₃₈N⁺]C₃₄H₃₅Cl NSCOO⁻+NaCl
Montelukast cetyl-pyridinium shows a solubility of 1.1 mg/mL in human serum, calculated as Montelukast sodium. It was measured by an HPLC method.

The results from the studies discussed in Examples 2 through 9 are summarized in Table 10. It is now clear from the examples provided that the sustained release compositions disclosed in the present invention possess better drug release pharmacokinetics compared to the immediate release composition.

As shown in Table 10, all of the sustained release compositions provided in the examples have a significantly longer duration of effect compared to the immediate release composition, 8 hours vs. 24-90 hours. This will allow for a less frequent administration of the medication for long term treatment. It will also allow a more stable drug concentration profile in blood plasma or serum because the sustained release composition can avoid the peaks-valleys profile seen in the immediate release composition where multiple injections are needed everyday.

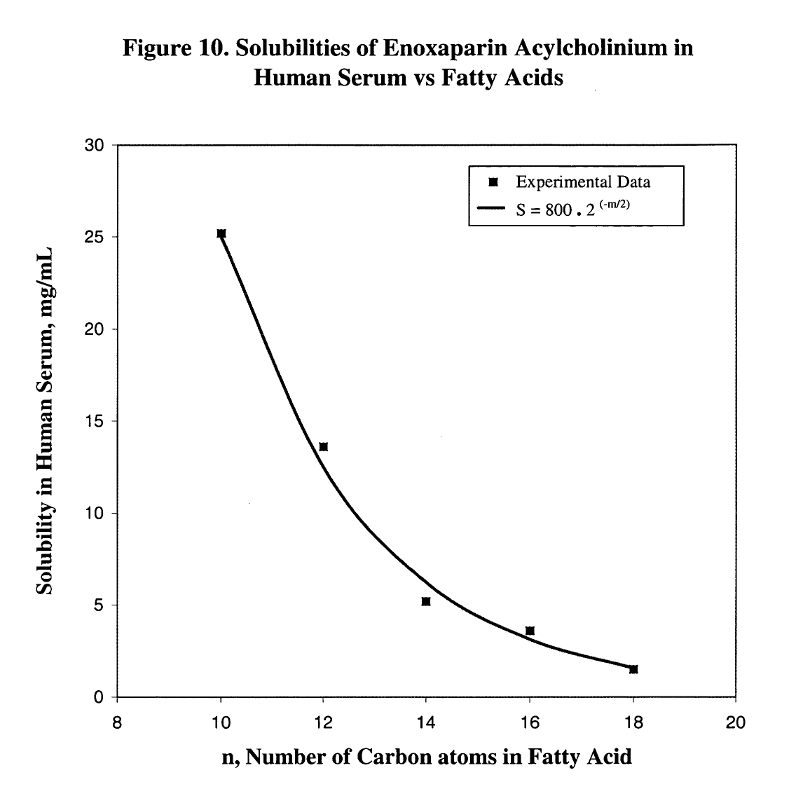

Table 10 also shows that the duration of the medication is dependent upon the number of carbon atoms or the type of cholinium selected. The higher the number of carbon atoms used in the composition, the longer the duration of effect. This is because the number of carbon atoms in the composition affects the solubility of the drugs in human serum. FIG. 10 shows the profile of enoxaparin acylcholinium solubilities in human serum as a function of the number of carbon atoms in fatty acids. This diagram shows that solubility of enoxaparin acylcholinium in human serum decreases as the number of carbon atoms increases by the following formula:
S=800·2^{-m/2}

Where S is the solubility of Enoxaparin acylcholinium in human serum (as mg/mL of enoxaparin sodium), and m is the number of carbon atoms in fatty acid (m=10˜18), which forms the acylcholinium.

TABLE 10
Summary of Study for Enoxaparin Acylcholinium

| | | | | | | | | | | | Relative | |
|---|---|---|---|---|---|---|---|---|---|---|---|---|
| | | Carbon | | Admini- | | | | | Amax | AUC | AUC | |
| | | Atoms | Solubility | stration | # of | Total Dose | Relative Dose | | of Xa | IU/mL | IU/mL | Duration |
| Examples | Cholinium | m | S, mg/mL | Method | Rats | D, mg/rat | mg/kg | mg/rat/12 hrs | IU/mL | hr | hr/mg | hrs |
| 1 | — | — | ˜300 | SC | 13 | 1 | 4 | 1 | 2.3 | 9.6 | 9.6 | 8 |
| 2 | Caproylcholinium | 10 | 25.2 ± 2.6 | SC | 6 | 6 | 24 | 3.3 | 2.9 | 47 | 7.9 | 24 |
| 3 | Laurylcholinium | 12 | 13.6 ± 2.2 | SC | 6 | 6 | 24 | 1.5 | 1.1 | 41 | 6.9 | 54 |
| 4 | Stearylcholinium | 18 | 1.5 ± 0.3 | SC | 4 | 6.6 | 26 | 1.4 | 1.3 | 35 | 5.3 | 57 |
| 5 | Carpoylcholinium | 10 | 25.2 ± 2.6 | IM | 6 | 6.6 | 26 | 1.9 | 2.5 | 49 | 7.3 | 42 |
| 6 | Laurylcholinium | 12 | 13.6 ± 2.2 | IM | 5 | 6.6 | 26 | 1.1 | 1.8 | 76 | 11.5 | 69 |
| 7 | Myristocholinium | 14 | 5.2 ± 2.0 | IM | 6 | 6.6 | 26 | 1.4 | 1.8 | 56 | 8.5 | 57 |
| 8 | Palmitoylcholinium | 16 | 3.6 ± 1.2 | IM | 6 | 4.8 | 19 | 0.9 | 1.6 | 58 | 12.0 | 66 |
| 9 | Streaylcholinium | 18 | 1.5 ± 0.3 | IM | 6 | 6.6 | 26 | 0.9 | 1.3 | 76 | 11.5 | 90 |

Note:
m: Number of Carbon Atoms in fatty acid for acylcholinium
S: Solubility of enoxaparin acylcholinium in human serum, as mg/mL of enoxaparin
Duration is based on a rat model and is measured as the time when anti-factor Xa in rat plasma > 0.2 IU/mL

Various methods of preparing the composition of the present invention are shown in FIG. 11.

While this invention has been described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention.

In particular, it is to be understood that this invention is not limited to the particular methodology, protocols, cell lines, animal species or genera, constructs, and reagents described as such may vary, as will be appreciated by one of skill in the art. For example, although a preferred embodiment of the present invention is directed to compositions comprising a salt or salts of quaternary ammonium of heparin or LMWH, for use in treating/preventing venous thrombosis and pulmonary embolism, the invention is more generally directed to salts of quaternary ammonium of any acid drug in the form of a suspension or an emulsion, wherein the quaternary ammonium salt provides for sustained release of the drug. The scope of the invention is, therefore, indicated by the following claims rather than by the foregoing description. All changes, modifications, and variations coming within the meaning and range of equivalency of the claims are to be considered within their scope.

What is claimed is:

1. A sustained release pharmaceutical composition, comprising a quaternary ammonium salt of an acid drug.

2. The composition of claim 1, wherein the acid drug is an organic compound with one or more acid groups selected from the group consisting of carboxyl group, sulfonic group, sulfate group, and mixtures thereof.

3. The composition of claim 1, wherein the acid drug is heparin.

4. The composition of claim 1, wherein the acid drug is low molecular weight heparin.

5. The composition of claim 4, wherein the low molecular weight heparin is a metal salt of a drug selected from the group consisting of adreparin, certoparin, dalteparin, enoxaparin, nadroparin, parnaparin, reviparin, and tinzaparin.

6. The composition of claim 1, wherein the quaternary ammonium salt comprises at least eight carbon atoms.

7. The composition of claim 1, wherein the quaternary ammonium salt is selected from the group consisting of choline esters, alkylpyridines, and their derivatives.

8. The composition of claim 1, wherein the quaternary ammonium salt comprises a choline esterified with an aliphatic acid or its derivatives.

9. The composition of claim 8, wherein the aliphatic acid comprises from three to ten carbon atoms.

10. The composition of claim 8, wherein the aliphatic acid is selected from the group consisting of capric acid, nonanoic acid, octanoic acid, heptanoic acid, hexanoic acid, pentanoic acid, butyric acid, propionic acid, and mixtures thereof.

11. The composition of claim 1, wherein the quaternary ammonium salt comprises a choline esterified with a fatty acid or its derivatives.

12. The composition of claim 11, wherein the fatty acid comprises an even number of carbon atoms between twelve to twenty two.

13. The composition of claim 11, wherein the fatty acid is selected from the group consisting of lauric acid, myristic acid, palmitic acid, stearic acid, arachidic acid, behenic acid, palmitoylleic acid, oleic acid, ricinoleic acid, linoleic acid, arachidonic acid, and mixtures thereof.

14. The composition of claim 1, wherein the quaternary ammonium salt comprises a choline esterified with a phosphatidic acid or its derivatives.

15. The composition of claim 1, wherein the quaternary ammonium salt comprises an ester of choline and alkylpyridine.

16. The composition of claim 1, wherein the quaternary ammonium salt of an acid drug has a solubility in human plasma or serum of less than or equal to about 50 mg/mL.

17. The composition of claim 1, wherein the quaternary ammonium salt of an acid drug is suspended in an aqueous medium.

18. The composition of claim 17, wherein the quaternary ammonium salt of an acid drug has a particle size less than or equal to about 200 micrometer.

19. The composition of claim 17, wherein the aqueous medium comprises water for injection, U.S.P.

18. The composition of claim 17, further comprising one or more inactive pharmaceutical excipients.

19. The composition of claim 18, wherein the excipient is selected from the group consisting of surfactants, suspending agents, dispersing agents, emulsifying agents, tonicity agents, preservatives, pH buffers, and mixtures thereof.

20. The composition of claim 17, wherein the pH is from about 5.0 to about 9.5.

21. A sterile pharmaceutical aqueous suspension or emulsion composition for parenteral administration, comprising a salt obtained by reacting an acid drug with a quaternary ammonium compound, the resulting salt having a longer sustained release in vivo than the acid drug.

22. The composition of claim 21, wherein the quaternary ammonium compound is an acylcholine.

23. The composition of claim 21, wherein the quaternary ammonium compound is an alkylpyridine.

24. The composition of claim 21, wherein the acid drug is heparin.

25. The composition of claim 21, wherein the acid drug is low molecular weight heparin.

26. A method for increasing the in vivo duration of a parenteral drug having one or more acid groups, comprising the step of reacting the drug with a quaternary ammonium compound in an aqueous medium to form a salt.

27. The method of claim 26, wherein the quaternary ammonium compound is an acylcholine.

28. The method of claim 26, wherein the quaternary ammonium compound is an alkylpyridine.

29. The method of claim 26, wherein the acid drug is heparin.

30. The method of claim 26, wherein the acid drug is low molecular weight heparin.

31. A sterile pharmaceutical aqueous suspension or emulsion composition for parenteral administration, comprising a salt obtained by reacting an acid drug with a quaternary ammonium compound, the resulting salt having a longer sustained release in vivo than the acid drug, wherein the salt is provided as an emulsion or suspension in pre-filled syringes, vials, or similar containers.

32. A sterile pharmaceutical aqueous suspension or emulsion composition for parenteral administration, comprising a salt obtained by reacting an acid drug with a quaternary ammonium compound, the resulting salt having a longer sustained release in vivo than the acid drug, wherein a solution of soluble salts of the acid drug is provided separately with a solution of soluble salts of the quaternary ammonium compound and the two solutions are combined prior to use to form the sustained release salt.

33. A sterile pharmaceutical aqueous suspension or emulsion composition for parenteral administration, comprising a salt obtained by reacting an acid drug with a quaternary ammonium compound, the resulting salt having a longer sustained release in vivo than the acid drug, wherein the acid drug is provided separately as a solid and combined with a solution of soluble salts of the quaternary ammonium compound prior to use to form the sustained release salt.

34. A sterile pharmaceutical aqueous suspension or emulsion composition for parenteral administration, comprising a salt obtained by reacting an acid drug with a quaternary ammonium compound, the resulting salt having a longer sustained release in vivo than the acid drug, wherein the quaternary ammonium compound is provided separately as a solid and combined with a solution of soluble salts of the acid drug prior to use to form the sustained release salt.

35. A sterile pharmaceutical aqueous suspension or emulsion composition for parenteral administration, comprising a salt obtained by reacting an acid drug with a quaternary ammonium compound, the resulting salt having a longer sustained release in vivo than the acid drug, wherein the acid drug and quaternary ammonium compound is provided as solid, which is combined with a diluent prior to use.
